(19) United States
(12) Patent Application Publication
(10) Pub. No.: US 2026/0191891 A1
(43) Pub. Date: Jul. 9, 2026

(54) DIETARY FIBRE COMPOSITION

(71) Applicant: MYOTA GMBH, Berlin (DE)

(72) Inventors: Thomas Jerome GURRY, Cologny (CH); Caitlin HALL, London (GB)

(21) Appl. No.: 19/132,424

(22) PCT Filed: Nov. 30, 2023
(86) PCT No.: PCT/EP2023/083806

§ 371 Date: May 23, 2025

(30) Foreign Application Priority Data
Nov. 30, 2022 (EP) 22210419.2

(51) Int. Cl. A61K 31/702; A61K 31/715; A61K 31/716; A61K 31/718; A61K 31/733; A61K 31/736; A61K 36/899; A61P 1/00; A61P 5/48; A61P 25/22

(52) U.S. Cl. CPC A61K 31/702; A61K 31/715; A61K 31/716; A61K 31/718; A61K 31/733; A61K 31/736; A61K 36/899; A61P 1/00; A61P 5/48; A61P 25/22

(57) ABSTRACT

The present invention relates to a dietary fibre mix having a composition tailored to be fermentable by the diverse microbiota profiles of diverse individuals, thus bringing health benefits associated with the production of short chain fatty acids. It also relates to nutritional compositions, such as food products or beverages, comprising such dietary fibre mix composition.

### TECHNICAL FIELD

The present invention relates to a dietary fibre mix having a composition tailored to be fermentable by the diverse microbiota profiles of diverse individuals, thus bringing health benefits associated with the production of short chain fatty acids. It also relates to nutritional compositions, such as food products or beverages, comprising such dietary fibre mix composition.

### BACKGROUND ART

Dietary fibres are natural essential components of many vegetal nutrients, such as fruits, vegetables, cereals and grains, and are also found in milk. Dietary fibres are commonly added to a large variety of food products. They are fermented by the microbiota of the individual consuming them. Upon fermentation by microbiota, metabolites are produced, in particular short chain fatty acids such as acetate, propionate and butyrate, which in turn provide numerous health benefits to the consumer.

Benefits associated with the consumption of dietary fibres are for example described in Liu et al., Butyrate: A Double-Edged Sword for Health?, Adv Nutr 2018; 9:21-29 and include:
a) improved bowel function,
b) improved post-prandial glycemic responses;
c) lower blood cholesterol;
d) improved intestinal homeostasis
e) improved energy metabolism
f) prevention of obesity and overweight
g) impact on insulin sensitivity;
h) type 2 diabetes prevention and treatment;
i) anti-inflammatory properties;
j) enhanced intestinal barrier function;
k) enhanced mucosal immunity; and
l) impact on the gut-brain axis.

Dietary fibres are commonly added to diverse nutritional compositions to impart diverse health benefits. In many prior art documents, long lists of possible fibers are provided, among which one or more fibre is/are to be chosen. This is the case for example the case of EP3513665A1, which discloses a dietary supplement comprising a prebiotic, together with other types of ingredients. The prebiotic is preferably a soluble food fibre, which may be selected from the group consisting of maltodextrin, inulin, oligofructose, lactose, lactulose, resistant starch, fructooligosaccharides, (FOS), galactooligosacchardes (GOS), transgalactooligosaccharides (TOS), wheat bran-derived arabinoxylooligosaccharides (AXOS), xylooligosaccharides (XOS), isomaltooligosaccharides (IMO), gentiooligosaccharides (GTO), glucooligosaccharides, pectic oligosaccharides, soy oligosaccharides, lactosucrose, polydextrose, sugar alcohols, alpha glucan, beta glucan, wheat dextrin, guar gum flaxseed gum, fenugreek gum, acacia gum, psyllium, and raffinose. Preferably the prebiotic is maltodextrin. The said supplement is disclosed as providing benefits in terms of weight loss and/or weight maintenance. All examples comprise maltodextrin as prebiotic. Beyond the specific pointer to maltodextrin, this document does not provide any suggestion of any particular fibre combination that would be particularly beneficial.

US2011/027412A1 discloses compositions comprising dietary fibres and more particularly a combination of gelling and non-gelling fibres. Such compositions are disclosed as promoting gastrointestinal and/or cardiovascular health. Non-limiting examples of gelling dietary fibers include psyllium, non-modified pectins, mannans (such as guar gum, locust bean gum, konjac, xanthan gum, glucomannans, galactomannans), beta-glucans, arabinans, galactans, aligns, agar, propylcelluloses and methylcelluloses (such as hydroxypropylmethyl cellulose and carboxymethyl cellulose), gelling carrageenans, and combinations thereof. Non-limiting examples of non-gelling dietary fibres include inulin, gum Arabic (acacia gum), larch arabinogalactan, resistant dextrin, high methyl pectin, cellulose, raffinose, stachyose, oligosaccharides (such as fructooligosaccharides, soy oligosaccharides, galactooligosaccharides, gentioligosaccharides, xylooligosaccharides, isomaltooligosaccharides, and arabinoxylanoligosaccharides, lactulose, hydrolysed guar gum, lignins, brans and grain fibers (such as oat hull fibre), oat bran, wheat bran, rice bran, non-gelling carrageenans, retrograded starch, resistant starch, slow digesting starch, resistant maltodextrins, sugar beet fibre, oilseed fibres such as from flax, and combinations thereof. Specific examples are presented with mixtures of inulin, psyllium and oat hull fibre.

Another example is EP1692949A2, which disclose a water-soluble dietary fibre-containing composition that can be incorporated into various food products and impart physiological effects, such as inhibiting an increase in the blood sugar level and an effect of regulating intestinal functions of consumers consuming the food containing the fibre-containing composition. The disclosed fibres include the water-insoluble fibres cellulose, wheat bran, fibres originated from apples, fibres derived from sweet potatoes and chitin and the water-soluble fibres pectin, powdered konjak (mannan), alginic acid salts, propylene glycol ester of alginic acid, guar gum, agar, hardly digestible dextrin, polydextrose, branched maltodextrin, inulin and guar gum hydrolysates. Also mentioned are oligosaccharides, such as fructo-oligosaccharides, galacto-oligosaccharides and xylo-oligosaccarides. In the examples only the following combinations are disclosed
a. hardly digestible dextrin with alginic acid salts (such as sodium alginate)
b. hardly digestible dextrin with propylene glycol ester of alginic acid
c. hardly digestible dextrin with agar
d. polydextrose with alginic acid salts
e. branched maltodextrin with alginic acid salts.
f. Hardly digestible dextrin, sodium alginate or agar and resistant starch.

It is further known from the prior art that different bacterial strains have different fermentation abilities and that not all fibres can be fermented by all types of bacteria present in the microbiota of an individual. It is also known that microbiota can vary from one individual to another. Nevertheless no prior art document addresses the problem of providing a mix of dietary fibres that would contain fibres that can be optimally fermented by most or all of the many different microbiota environments found in different individuals. The present invention aims at solving this problem.

### SUMMARY OF INVENTION

In a first aspect, the invention relates to a dietary fibre composition comprising
a mixture of the following dietary fibres:
   a. isolated fructo-oligosaccharides (FOS);
   b. isolated inulin;
   c. isolated resistant maltodextrin;
   d. isolated resistant dextrin;
   e. isolated galactomannan polysaccharide; and
   f. isolated partially hydrolysed galactomannan.

In a second aspect, the invention relates to a nutritional composition comprising the dietary fibre composition of the present invention.

In a third aspect, the invention relates to a process for producing a nutritional composition according to the present invention, comprising admixing the dietary fibre composition of the invention with the other ingredients of the nutritional composition.

In a fourth aspect, the invention relates to a dietary fibre composition according to invention or to a nutritional composition of the invention, for use in therapy.

### BRIEF DESCRIPTION OF DRAWINGS

FIG. 1 Butyrate production rates (in millimolar per hour) calculated for each fermentable substrate from ex vivo fermentation experiments on stool samples from volunteers. Error bars represent standard deviations to illustrate inter-individual variability. Number of measurements differ by fibre and can be found in the Materials and methods section of Example 1.

FIG. 2 Propionate production rates (in millimolar per hour) calculated for each fermentable substrate from ex vivo fermentation experiments on stool samples from volunteers. Error bars represent standard deviations to illustrate inter-individual variability. Number of measurements differ by fibre and can be found in the Materials and methods section of Example 1.

FIG. 3 Expected average fermentation profiles of each mix based on the volunteers in our database. Butyrate production rate is shown in black, while propionate production rate is shown in grey.

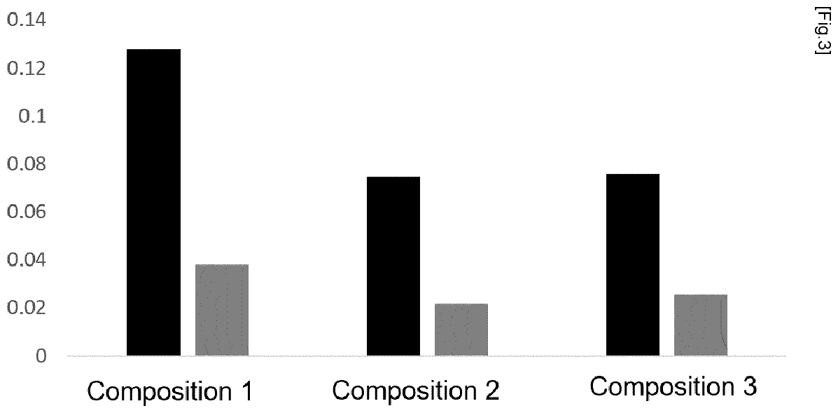

FIG. 4 Results from HbA1c, insulin sensitivity (ISI-OGTT) and fasting insulin measures. Change from baseline to week 16 in (B) HbA1c within a sub-group including participants with baseline HbA1c levels<6.0%; (C) insulin sensitivity (assessed using ISI-OGTT); and (D) fasting blood insulin (μU/mL) between Intervention (dark grey), and Placebo (light grey).

FIG. 5 Difference in gut microbiome diversity and richness scores between participants consuming the fibre mix of the invention (series labelled as “Yes”) and participants who did not consume the fibre mix (series labelled as “No”). * denotes pcorr<0.05.

FIG. 6 Results from stress and gut symptom scores on completed participants (N=37). Change from baseline to week 12 in (A) Perceived Stress Scores (PSS); (C) Total Gastrointestinal Symptom Rating Scale (GSRS) scores for the Intervention (light grey) and Control (dark grey) groups. * denotes p_{corr}<0.05.

### DETAILED DESCRIPTION

### Dietary Fibre Composition

The dietary fibre composition of the present invention comprises a combination of the fibres that are best fermented across individuals having different microbiota, thus generating the highest production rate of butyrate and propionate.

As the microbiota differs from one individual to another, the fibres that are best fermented are also different across individuals. The present inventors have assessed the short chain fatty acid production rate upon fermentation of a large range of fibres by a high number of different microbiota and have identified the fibres that were best fermented across the whole population of subjects, in order to provide sufficient short chain fatty acids metabolites. On that basis, they developed a dietary fibre composition that ensures that sufficient short chain fatty acids can be timely produced upon consumption of the composition by any subject and fermentation of the dietary fibre composition by the microbiota of such subject. The dietary fibre mix composition is therefore advantageous in that it contains all fibres required to allow for sufficient production of short chain fatty acid, in particular butyrate and propionate, in a wide variety of individuals. Compared to other mixes, it is optimized to fit the microbiota of most of the population of consumers, as a one-fits-all composition.

The mandatory components of the dietary fibre composition are fructo-oligosaccharides, inulin, resistant maltodextrin, resistant dextrin, galactomannan polysaccharides and partially hydrolysed galactomannan. Optionally, the dietary fibre composition also comprises galacto-oligosaccharides. The dietary fibre composition is based on such fibres in isolated form, i.e. these are not provided in the form of a complex vegetable source containing such fibres. The term “isolated” however does not exclude the presence of minor amounts or traces of other compounds, such as impurities that were not removed by the purification process or side products obtained during the fibre synthesis. The term isolated also does not exclude that several of the above-listed fibres be present in combination is one single ingredient, provided that such fibre source contains substantially only fibres among those above-listed. This can be for example the case for an isolated fructan ingredient that is a mixture of inulin and fructo-oligosaccharides.

It is advantageous that the fibres be in isolated form, for the purpose of allowing to control the amount of the required fibres present in the composition. When fibres are provided in the form of complex matrices, such as a natural source, for example a cereal, it is often complicated to determine whether the desired fibre is present and in which amount it is present in such matrix. As the present fibre composition has been tailored to fit the widest variety of microbiota types, it is important that no fibre is missing from the mix and preferably it is also sought to ensure that each fibre is present in the preferred amounts recited herein. For this purpose, isolated fibres are used in the present dietary fibre composition.

In addition, the fibre composition may preferably also contain a source of arabinoxylan and/or a source of beta-glucan. Such source can be either arabinoxylan and/or a source of beta-glucan in isolated form or a more complex ingredient comprising arabinoxylan and/or a source of beta-glucan, such as for example oat and/or wheat fibre.

Fructo-oligosaccharides (FOS) and inulin are both fructans. Inulin is a polymer of D-fructose residues linked by γ(2→1) bonds with a terminal α(1→2) linked D-glucose. Inulin has the general structure Glu-Fruₙ (or GFₙ), wherein n is 10 to 60 (degree of polymerisation of 10 to 60). FOS are oligosaccharides having the general structure Glu-Fruₙ (or GFₙ), wherein n is 2 to 7 (degree of polymerisation of 2 to 7). The fructose units may be linked by diverse linkage, including β(2→1) and β(1→2). Both inulin and FOS are non-digestible and are therefore not degraded by the enzymes in the digestive tract of an individual consuming them. As such, they are available for fermentation by the microbiota of such individual.

It is particularly advantageous that the composition comprises both FOS and inulin because a combination of fructans of different length results in increased SCFA production across different individuals compared to a single length. This applies to both butyrate and propionate production.

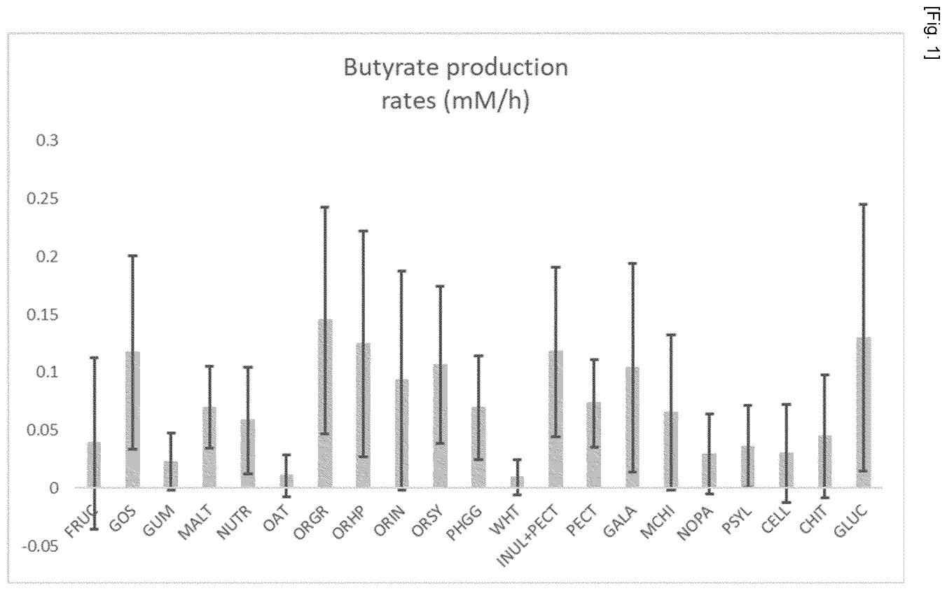

The present inventors have identified that mixture of FOS and inulin were among the highest butyrate and propionate producers among the tested fibres ([FIG. 1]).

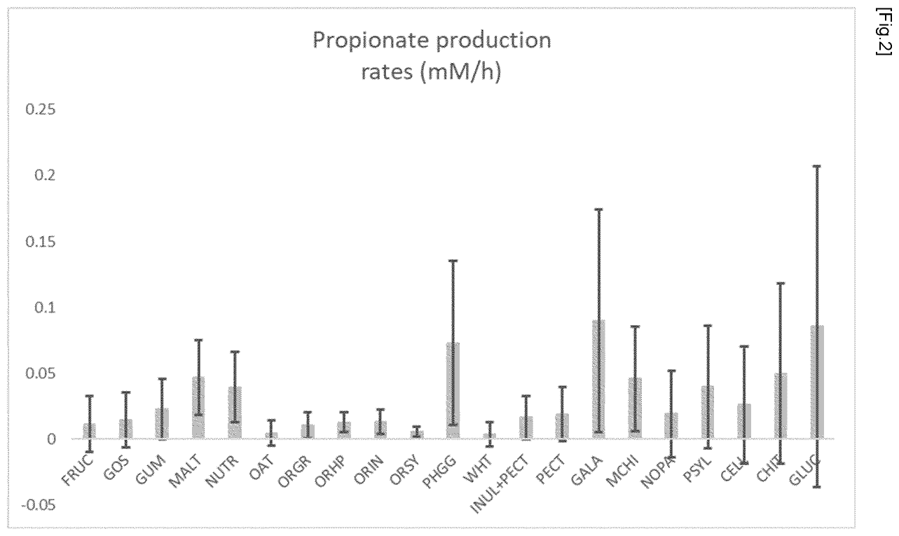

Resistant maltodextrin is a digestion-resistant dietary fibre that is soluble and non-viscous. It is an oligosaccharide formed of 3 to 20, preferably 3-17 glucose units bound by α(1→4) linkages formed by a process of ‘retrogradation’, where the glucose chains (amylose and amylopectin) realign after being heated and cooled and has a chemical formula C(₆ₙ)H(₁₀ₙ₊₂)O(₅ₙ₊₁). Resistant maltodextrin is advantageous because its fermentation produces butyrate with a low inter-individual variation, compared to most other dietary fibres. The mean propionate production upon its fermentation by diverse microbiota is also higher than most other tested dietary fibres (see [FIG. 2]

Resistant dextrin is a digestion-resistant dietary fibre formed of glucose units mainly bound by α(1→4) or α(1→6) linkages having a chemical formula (C₆H₁₀O₅) n and branched through other linkages, such as β(1→6), β(1→2), α(1→6) and α(1→2). Its branched structure is responsible for its property of resisting to digestive enzymes. Resistant dextrin is typically obtained by partial enzymatic hydrolysis of starch, followed by degradation by heat under acidic conditions. The latter step is responsible for the branching of the structure and confers to dextrin its resistance to digestion.

The mean butyrate production rate upon fermentation of resistant maltodextrin, resistant dextrin by diverse microbiota is advantageously higher than other fibres such as chitosan, microchitosan, psyllium, cellulose and NOPA. In addition, inter-individual variation is lower than with many other fibres, as can be seen from the short error bar associated with the resistant maltodextrin and resistant dextrin histograms on [FIG. 1] and [FIG. 2]. Namely, these fibres were metabolised into butyrate by the microbiota from all donors used in Example 1 which makes these fibres particularly advantageous for use in a food product for use in a wide diversity of subjects.

Galactomannan polysaccharides are composed of a mannose backbone consisting of mannose units linked by an α(1→4) linkage, branched with 1-6-linked galactose side groups. The diverse galactomannan polysaccharides differ in their mannose to galactose ratio. Examples of galactomannan polysaccharides include fenugreek gum (having a mannose:galactose ratio of about 1:1), guar gum (having a mannose:galactose ratio of about 2:1), tara gum (having a mannose:galactose ratio of about 3:1), locust bean gum (having a mannose:galactose ratio of about 4:1) and cassia gum (having a mannose:galactose ratio of about 5:1). Preferably, the galactomannan has a mannose:galactose ratio of 1:1 to 3:1, more preferably of 1.5:1 to 2.5:1. Most preferably, the galactomannan is guar gum.

Partially hydrolysed galactomannan is a dietary fibre obtained by controlled enzymatic hydrolysis of a galactomannan polysaccharide such as described above. Preferably the hydrolysed galactomannan is a hydrolysed galactomannan having a mannose:galactose ratio of 1:1 to 3:1, more preferably of 1.5:1 to 2.5:1. Most preferably, it is partially hydrolysed guar gum.

Galactomannan polysaccharides and partially hydrolysed galactomannan are particularly advantageous for their ability to be metabolized to propionate by the microbiota from the donors of Example 1. Indeed, among the fibres tested in Example 1, galactomannan polysaccharides and partially hydrolysed galactomannan (partially hydrolysed guar gum) provided the highest mean propionate production rates. In addition, even though the inter-individual variation in propionate production rates is high for both galactomannan polysaccharides and partially hydrolysed galactomannan, these fibres are advantageously fermented to propionate in all subjects, at least to some extent. Only glucose provided as high propionate production rates as galactomannan polysaccharides, however with a higher inter-individual variation and even absence of propionate production by the microbiota of part of the donors of Example 1. In addition to their outstanding ability to be metabolized to propionate, galactomannan polysaccharides and partially hydrolysed galactomannan exhibit good butyrate production rates, in particular galactomannan polysaccharides, and are characterized by a limited inter-individual variability, as the microbiota of all donors allowed for butyrate production upon fermentation of galactomannan polysaccharides and of partially hydrolysed galactomannan.

Galacto-oligosaccharides are polymers of galactose units, optionally with a terminal glucose unit, formed by the polymerisation of lactose. The chain length and the type of linkage between the galactose units varies significantly between different Galacto-oligosaccharide fractions, namely depending on the enzyme involved in the polymerisation of lactose.

Galacto-oligosaccharides are very advantageous, due to the high mean butyrate production rate upon fermentation by the microbiota of diverse donors. Galacto-oligosaccharides are however optional in the composition of the present invention because galacto-oligosaccharide ingredients may contain traces of lactose, which may not be well tolerated by a few consumers facing adverse health conditions. Therefore, this dietary fibre is omitted in compositions targeting fragile consumers or consumers with lactose intolerance.

The optional arabinoxylan and/or beta-glucan source can the purified fibre or any ingredient containing such fibres, preferably it is a fibre composition such as oat and/or wheat fibre. Arabinoxylan and beta-glucan are both already known for their beneficial effect on improving bowel function, post-prandial glycaemic responses and their ability to lower blood cholesterol. These fibres are therefore advantageously used in combination with the mandatory components of the dietary fibre composition of the present invention, optionally with galacto-oligosaccharides.

In a preferred aspect, the dietary fibres are present in the dietary fibre composition of the present invention in the following amounts:
a. fructo-oligosaccharides are present in an amount of 10 to 50 wt %, preferably 10 to 40 wt %, more preferably 14 to 34 wt %, based on the total weight of the composition;
b. inulin is present in an amount of 5 to 40 wt %, preferably 8 to 30 wt %, more preferably 10 to 26 wt %, based on the total weight of the composition;
c. resistant maltodextrin is present in an amount of 3 to 30 wt %, preferably 3 to 25 wt %, more preferably 5 to 20 wt %, most preferably 5 to 15 wt %, based on the total weight of the composition;
d. resistant dextrin is present in an amount of 2 to 20 wt %, preferably 3 to 15 wt %, more preferably 4 to 12 wt %, based on the total weight of the composition;
e. the galactomannan polysaccharide is present in an amount of 0.5 to 10 wt %, preferably 1 to 7 wt %, more preferably 1 to 5 wt %, based on the total weight of the composition; and
f. partially hydrolysed galactomannan is present in an amount of 1 to 20 wt %, preferably 2 to 15 wt %, more preferably 4 to 13 wt %, most preferably 5 to 12 wt %, based on the total weight of the composition.

When present, the optional dietary fibres are present in the following amounts:
a. galacto-oligosaccharides are preferably in an amount of 5 to 25 wt %, preferably 8 to 20 wt %, more preferably 10 to 15 wt %, based on the total weight of the composition
b. the at least one source of arabinoxylan and/or at least one source of beta-glucan are preferably in a total amount of 35 to 55 wt %, preferably 40 to 50 wt %.

When present, wheat fibre is preferably in an amount of 15 to 35 wt %, preferably 20 to 30 wt %, based on the total weight of the composition and oat is preferably in an amount of 10 to 25 wt %, preferably 15 to 23 wt %, based on the total weight of the composition.

In a particular aspect, the dietary fibre composition of the present invention consists of fructo-oligosaccharides, inulin, resistant maltodextrin, resistant dextrin, galactomannan polysaccharides, partially hydrolysed galactomannan, optionally galacto-oligosaccharides, optionally a source of arabinoxylan and optionally a source of beta-glucan. Preferably, the dietary fibres composition consists of such ingredients in the amounts disclosed above.

### Nutritional Composition

In an embodiment, the invention relates to a nutritional composition comprising a dietary fibre composition such as described above. Such dietary fibre composition can be added to any kind of nutritional composition. In particular, the nutritional composition may be in the form of a nutritional supplement, of a food product or of a beverage.

A nutritional supplement typically comprises the dietary fibre composition of the present invention, together with a suitable carrier and optionally other ingredients. It may be in solid, liquid or semi-solid form, such as in the form of a powder, a liquid, a paste, a gel a tablet, a capsule or a pastille. The supplement can be consumed as such or added to a food product or drink, for example by dissolving or dispersing the supplement in a liquid or by sprinkling on food.

Any liquid or solid carrier suitable for oral administration can be used. Examples of such carriers include solvents or diluents, in particular water or aqueous solutions and oils, gelatine, gums or solid organic or inorganic carriers, such as talc, sugars, starch or gum arabic. The supplement may further contain emulsifying, dispersing or solubilizing agents (such as oils, fats, waxes, lecithin etc.), hydrocolloids (such as gums, proteins, modified starches), binders, film forming agents, encapsulating agents, wall/shell materials, coatings, adsorbents, fillers, wetting agents, flowing agents, jellifying agents, gel forming agents, preservatives (such as antioxidants and antimicrobials), flavouring agents, stabilizers, buffers, lubricants and/or colorants.

The nutritional composition may also contain additional nutrients, such as macronutrients, vitamins, minerals or probiotics. In a specific aspect however, the nutritional composition does not comprise probiotics. Indeed, the present dietary fibre composition is made such as to fit diverse types of microbiota present in the gastrointestinal tract of consumer and to ensure sufficient short chain fatty acids production, so that further administration of probiotics is only possible but not necessary.

The nutritional composition can also be a beverage or a food product. The beverage or food product can for example be a milk, a smoothie, a soft-drink, an infant formula, a growing-up milk, a baby food, a cereal-based product such as bread, pastries, cake, breakfast cereals, muesli, granola or porridge, a soup, a dessert, a meat or alternative meat product, and the like.

The nutritional composition is typically produced by admixing a dietary fibre composition according to the invention with the other ingredients of the nutritional composition and preferably packed in a suitable way.

### Composition for Use in Therapy

The dietary fibre composition can advantageously be used in therapy, due to its property of being metabolized to short chain fatty acids upon fermentation by the microbiota of an individual consuming it. The effect is advantageously obtained for a wide variety of subjects having different microbiota, as demonstrated by the examples below.

The present invention therefore relates to a dietary fibre composition as described above, for use in therapy. Therapy is intended here as the treatment and/or the prevention of disease or disorder and/or the reduction of the severity and/or of the symptoms of a disease or disorder.

In a preferred embodiment of the present invention, the dietary fibre composition of the invention is for use in increasing the production rate of at least one short chain fatty acid (SCFA) in an individual. Preferably, the at least one SCFA is butyrate, propionate, acetate or mixtures thereof. In a more preferred embodiment, the SCFA is butyrate. More preferably, the individual is an individual in need of an increase in SCFA, respectively butyrate and/or propionate, production.

In other preferred embodiments, the dietary fibre composition of the present invention is for use in providing at least one of the following therapeutic effects:
a. improving bowel function in an individual consuming the dietary fibre composition or the nutritional composition;
b. reducing gastrointestinal symptoms in an individual consuming the dietary fibre composition or the nutritional composition;
c. increasing microbiota diversity in an individual consuming the dietary fibre composition or the nutritional composition;
d. improving post-prandial glycaemic responses in an individual consuming the dietary fibre composition or the nutritional composition;
e. lowering blood lipids, preferably blood cholesterol in an individual consuming the dietary fibre composition or the nutritional composition;
f. preventing elevation of blood cholesterol levels in an individual consuming the dietary fibre composition or the nutritional composition;
g. lowering blood glucose levels, preferably lowering glycated haemoglobin levels, more preferably lowering HbA1c, in an individual consuming the dietary fibre composition or the nutritional composition;
h. preventing elevation of blood glucose levels, preferably preventing elevation of glycated haemoglobin levels, more preferably preventing elevation of HbA1c, in an individual consuming the dietary fibre composition or the nutritional composition; preferably preventing elevation of blood glucose levels, preferably preventing elevation of glycated haemoglobin levels, more preferably preventing elevation of HbA1c in an individual consuming the dietary fibre composition or the nutritional composition having a diagnosis of pre-diabetes; even more preferably preventing elevation of blood glucose levels, preferably preventing elevation of glycated haemoglobin levels, more preferably preventing elevation of HbA1c in an individual consuming the dietary fibre composition or the nutritional composition having a diagnosis of pre-diabetes and having HbA1c levels below 6.0 before administration of the composition;
i. reducing blood pressure in an individual consuming the dietary fibre composition or the nutritional composition;
j. preventing the elevation of blood pressure in an individual consuming the dietary fibre composition or the nutritional composition;
k. improving intestinal homeostasis in an individual consuming the dietary fibre composition or the nutritional composition;
l. improving energy metabolism in an individual consuming the dietary fibre composition or the nutritional composition;
m. preventing obesity and/or overweight in an individual consuming the dietary fibre composition or the nutritional composition;
n. improving insulin sensitivity in an individual consuming the dietary fibre composition or the nutritional composition, preferably in such an individual having a diagnosis of pre-diabetes;
o. preventing, treating or reducing the severity of type 2 diabetes in an individual consuming the dietary fibre composition or the nutritional composition;
p. preventing, treating or reducing inflammation in an individual consuming the dietary fibre composition or the nutritional composition;
q. enhancing intestinal barrier function in an individual consuming the dietary fibre composition or the nutritional composition;
r. enhancing mucosal immunity in an individual consuming the dietary fibre composition or the nutritional composition;
s. preventing, treating or reducing the severity of infections, such as viral, bacterial or fungal infections, in an individual consuming the dietary fibre composition or the nutritional composition;
t. enhancing the bidirectional communication between the central and the enteric nervous system (gut brain axis) in an individual consuming the dietary fibre composition or the nutritional composition;
u. preventing or reducing the severity of autism-spectrum, anxiety and/or depressive behaviours in an individual consuming the dietary fibre composition or the nutritional composition;
v. preventing, treating and/or reducing the severity of irritable bowel syndrome in an individual consuming the dietary fibre composition or the nutritional composition;
w. reducing stress, preferably stress perception, in an individual consuming the dietary fibre composition or the nutritional composition; and/or
x. reducing fasting blood insulin levels in an individual consuming the dietary fibre composition or the nutritional composition, preferably in such an individual having a diagnosis of pre-diabetes.

In a preferred aspect, the therapeutic effect is achieved as a result of the increase of the SCFA production rate in the individual consuming the dietary fibre composition or the nutritional composition. Indeed, it has been established in the prior art that SCFA production, and in particular butyrate production was associated with such therapeutic effects.

In other words, the present invention relates to method of treatment comprising administering to a subject in need thereof a dietary fibre composition or a nutritional composition according to the present invention. In a preferred aspect, the method of treatment is for increasing the production rate of at least one short chain fatty acid (SCFA) in an individual. Preferably, the at least one SCFA is butyrate, propionate, acetate or a mixture thereof. In a more preferred embodiment, the SCFA is butyrate. In another preferred aspect, the method of treatment is for providing at least one of therapeutic effects mentioned above.

In still other words, the present invention relates to the use of a dietary fibre composition or of a nutritional composition according to the present invention in therapy. Preferably the use is for increasing the production rate of at least one short chain fatty acid (SCFA) in an individual. Preferably, the at least one SCFA is butyrate, propionate, acetate or a mixture thereof. In a more preferred embodiment, the SCFA is butyrate. In another preferred aspect, the use is for providing at least one of the therapeutic effects mentioned above.

In still other words, the present invention relates to the use of a dietary fibre composition or a nutritional composition according to the present invention for the manufacture of a medicament. In a preferred aspect, the medicament is for increasing the production rate of at least one short chain fatty acid (SCFA) in an individual. Preferably, the at least one SCFA is butyrate, propionate, acetate or a mixture thereof. In a more preferred embodiment, the SCFA is butyrate. In another preferred aspect, the medicament is for providing at least one of the therapeutic effects mentioned above.

In a preferred aspect, the dietary fibre composition or the nutritional composition of the present invention is administered in a therapeutically effective amount. Preferably, it is administered at a dose of at least 5 g, preferably from 5 to 30 g, of the dietary fibre composition per day.

There is no limit to the duration of administration of the dietary fibre composition according to the present invention. The dietary fibre composition or the nutritional composition is advantageously consumed on the long term to provide the above-described benefits for a prolonged duration. However, even short-term administration schemes provide benefits. For an optimal effect it is preferred that the dietary fibre composition or the nutritional composition be administered for a period of at least one week, preferably at least two weeks, preferably at least three weeks, preferably at least one month, more preferably at least two months, most preferably at least three months.

In a particular aspect, the individual is in need of any one or more of the above-described therapeutic effects.

In a preferred aspect, the individual has signs of pre-diabetes and/or suffers from overweight or obesity. More preferably the individual having signs of pre-diabetes is not clinically diagnosed with diabetes. In a preferred aspect, the individual having signs of pre-diabetes is characterized by haemoglobin A 1 C above 6.0%, more preferably between 6.0 and 6.5%, most preferably between 6.0 and 6.4% within the previous 12 months.

In another preferred aspect, the individual does not suffer from a severe hepatic diseases, such as chronic persistent hepatitis, liver cirrhosis and/or co-occurrence of positive hepatitis B virus surface antigen and abnormal hepatic transaminase (serum concentrations of alanine transaminase or aspartate transaminase >2.5 times the upper normal limit).

In another preferred aspect, the individual does not suffer from impaired microbiota or, if the individual suffers from impaired microbiota, the nutritional composition consumed by such individual further comprises at least one probiotic. In one aspect, the individual suffering from impaired microbiota is an individual subjected to a treatment known to impair microbiota. Treatments that are known to impair microbiota are for example:
a) the use antibiotics for more than 3 days within 3 months prior to administration;
b) the use of weight-loss drug within the last one months, preferably within the last two months, more preferably within the last three months prior to administration;
c) the occurrence of a gastrointestinal surgery (except appendicitis or hernia surgery) within the last one months, preferably within the last two months, more preferably within the last three months prior to administration;
d) the use of drug therapy to treat cholecystitis, peptic ulcers, urinary tract infection, acute pyelonephritis, urocystitis or hyperthyreosis.

In another preferred aspect, the individual does not suffer from pituitary dysfunction, severe organic diseases (including cancer, coronary heart disease, myocardial infarction or cerebral apoplexy), infectious diseases (including pulmonary tuberculosis and AIDS), significant dyslipidemia and/or hypertension above 160/100.

### EXAMPLES

### Example 1: Assessment of Fermentation Capabilities of Different Individuals

### Materials and Methods

### 1. Experimental Determination Fermentation Capability

### Volunteer Recruitment

Whole stool samples were collected from volunteers using stool collection hats and processed within 4 hours of passage. Volunteers were given the option either to donate a sample directly at the laboratory, or to request an at-home sampling kit and schedule a courier pick-up same morning. A total of N=71 volunteers were recruited.

### Sample Processing

Samples were homogenised into a faecal slurry by diluting the sample 2.5× in L-cysteine reduced PBS buffer solution and homogenising as well as filtering using WhirlPak filter bags. An aliquot from the faecal slurry was taken for DNA sequencing and the remainder was aliquoted onto a 96-well plate. Individual wells were then diluted a further 2× by adding either a control condition (i.e. PBS buffer) or the relevant spike-in (a dietary fibre dissolved in L-cysteine reduced PBS buffer) reaching a final spike-in concentration of 10 g/L. The different spike-ins were: fructo-oligosaccharides, galacto-oligosaccharides, gum arabic, inulin, resistant maltodextrin, resistant dextrin, oat fibre, partially hydrolysed guar gum, glucose, fructose, wheat fibre, pectin, nopal cactus fibre, galactomannan, psyllium husk, cellulose, chitosan and microchitosan. Two separate 500 μL samples (from distinct wells, constituting biological replicates) are taken at 0 h, 2 h and 4 h and frozen at −80° C. for subsequent analyses.

### GC-FID Measurements

Samples were removed from the freezer and allowed to thaw. The pH of the thawed faecal slurry samples was adjusted to 2-3 with a 1% aqueous sulfuric acid solution. The acidified samples were vortexed for 10 minutes and then centrifuged for 10 minutes at 10′000 rpm. Meanwhile, 200 μL of MeCN and 100 μL of a 0.1% v/v 2-methyl pentanoic acid solution were pipetted into screw neck vials. Once centrifugation finished, 200 μL aliquots of the clear supernatant were transferred in the screw neck vials. The tubes were shaken and analysed by GC-FID on a Perkin Elmer Clarus 500 with auto-sampler equipped with a flame ionisation detector and a hydrogen generator. A DB-FFAP column (30 m, 0.250 mm diameter, 0.25 μm film) was used for analyte separation. The temperature gradient used was as follows: 5 minutes at 100° C., then up to 200° C. over 10 minutes, followed by 5 minutes at 200° C. (20 minutes total runtime). Each Short Chain Fatty Acid concentration was calculated from the ratio of the signal intensity of each SCFA peak to the signal intensity of the internal standard of known concentration. SCFAs measured were acetate, propionate, butyrate, iso-butyrate, valerate, iso-valerate, caproate, iso-caproate, and hexanoate.

### Data QC

Each GC-FID analysis batch was quality controlled in the following manner: (i) visual inspection of the chromatograms; (ii) examination of the signal of the internal standard (2-methylpentanoic acid) for major discrepancies between samples in a batch, in which case the batch was thrown out and repeated; (iii) examination of QC samples at the beginning, middle and end of a run, consisting of samples of known SCFA concentrations (100 μM and 1 mM); (iv) removal of specific data points/replicates for which the coefficient of variation between concentration values in experimental replicates for a specific SCFA (after correction by internal standard) exceeded 20%.

### Calculation of Fermentation Capability

Experimental replicates for each timepoint were averaged and a production rate was calculated for each fibre and SCFA pair by computing the hourly production rate between 2 h and 4 h timepoints.

### Fibres Used in Fermentation Experiments

Fermentable substrates and fibres tested in fermentation experiments, with abbreviations used in plots contained in this document, are as follows. FRUC=fructose (Sigma; N=54); GOS=galacto-oligosaccharides (Tate & Lyle; N=27); GUM=gum arabic (Carl Roth; N=66); MALT=resistant maltodextrin (Fibersol-2; N=65); NUTR=resistant dextrin (Roquette; N=64); OAT=oat fibre (Sanacel; N=36); ORGR=Orafti GR fructo-oligosaccharide and inulin mix (Beneo; N=4); ORHP=Orafti HP fructo-oligosaccharide and inulin mix (Beneo; N=4); ORIN=Orafti inulin (Beneo; N=4); ORSY=Orafti sybergy fructo-oligosaccharide and inulin mix (Beneo; N=4); PHGG=partially-hydrolysed guar gum (Sunfiber AG; N=65); WHT=wheat fibre (Sanacel; N=34); PECT=pectin (Carl Roth; N=28); GALA=galactomannan (; N=6); MCHI=micro-chitosan (Nutricology; N=5); NOPA=nopal cactus fibre (Seagate; N=6); PSYL=psyllium husk (Solgar; N=6); CELL=cellulose (Nutricology; N=3); CHIT=chitosan (Unsure; N=3); GLUC=glucose (Sigma; N=3).

### 2. Microbiome Sequencing

Aliquots from the original faecal slurries were dissolved in nucleic acid stabilization buffer and sent for 16S rRNA sequencing using the 16S rRNA sequencing kits from Carbiotix AB (Lund, Sweden). The resulting FASTQ sequencing files are stored in our database for future analyses.

### 3. Plasma SCFA Analysis

### Sample Processing

20.4 μL of 1 mM hexanoic acid-d11 were added to 200 μL plasma aliquots, followed by deproteination by addition of 800 μL acidified MeCN (MeCN+0.1% formic acid). Solutions were vortex-mixed vigorously and centrifuged at 10′000 rpm for 10 minutes at 4° C. A 250 μL aliquot of the clear supernatant was transferred into an Eppendorf. Derivatisation was then carried out by addition of 250 μL of a stock solution containing 379.2 mg of 3-nitrophenylhydrazine (3-NPH), 115.1 mg of 1-ethyl-3-(3-dimethylaminopropyl) carbodiimid (EDCi) and pyridine (300 μL) in 50% aqueous MeCN (20 mL) followed by incubation at 40° C. for 30 minutes. 500 μL aliquots were transferred to screw neck vials for LC-MS analysis.

### SCFA Quantification by LC-MS

LC-MS analysis of derivatised plasma samples was performed on an Acquity UPLC I-class system (Waters) coupled to a Xevo TQ-S micro mass spectrometer (Waters) equipped with an electrospray ionization (ESI) source and operated in negative-ion mode. A Waters BEH C18 UPLC column (2.1×100 mm, 1.7 μm) equipped with a VanGuard Pre-Column 3/Pk using water:formic acid (100:0.01, v/v, solvent A) and acetonitrile:formic acid (100:0.01, v/v, solvent B) as mobile phases for gradient elution were used for liquid chromatographic separation of analytes. The gradient elution protocol used was 5%-65% B in 7.5 minutes, 65%-95% B in 0.1 minute, and then held at 95% B for 3 minutes. The column was equilibrated for 2.5 minutes at 5% B between injections.

### Results: Fermentation Capabilities of Different Individuals

Measuring the fermentation capability of an individual involved computing the production rate of each SCFA from each fibre spike-in (cf. Methods). [FIG. 1] and FIG. 2 show the average production rates of Butyrate and Propionate, respectively, which are considered as the most important SCFA in view of most health benefits. Error bars represent standard deviations, as a measure of the variance between individuals.

### Example 2: Dietary Fibre Compositions According to the Invention

Three different compositions dietary fibre compositions according to the present invention are provided in [Table 1] below.

TABLE 1

| | Concentration | Concentration | Concentration |
|---|---|---|---|
| | in Comp. 1 | in Comp. 2 | in Comp. 3 |
| Dietary Fibre | (g/100 g) | (g/100 g) | (g/100 g) |
| Wheat fibre (WHT) | 0.0 | 26.0 | 26.0 |
| Oat fibre (OAT) | 0.0 | 18.0 | 18.0 |
| Galacto-oligosaccharides (GOS) | 0.0 | 13.8 | 0.0 |
| Fructo-oligosaccharides (FOS) | 33.2 | 14.0 | 18.6 |
| Inulin (INUL) | 25.9 | 11 | 14.5 |
| Resistant maltodextrin (MALT) | 13.8 | 5.8 | 7.7 |
| Partially-hydrolysed guar gum (PHGG) | 11.9 | 5.0 | 6.7 |
| Resistant dextrin (NUTR) | 11.1 | 4.7 | 6.2 |
| Guar gum (GUM) | 4.1 | 1.7 | 2.3 |

Composition 1 was designed as an immunity regulator and focuses on customers with IBD. In order to optimize the anti-inflammatory properties of the composition, it was designed by combining the fibres that provided the highest butyrate production across different individuals. It contains no insoluble fibres and no GOS (which is well fermented but contains significant quantities of lactose and can induce lactose intolerance-induced inflammation).

Composition 2 is designed as a metabolic regulator and combines soluble, insoluble and fermentable fibres. It contains the already available EFSA health claims of the insoluble fibres (i.e. WHT and OAT), together with an optimized fermentability profile for boosting butyrate and propionate production.

Composition 3 is designed as a mix for improving mental wellness. It combines the benefits of insoluble fibres with soluble, fermentable fibres. It does not contain any GOS (which contains lactose), to avoid lactose intolerance-induced irritability.

In all cases, after the relevant constraint was introduced (e.g. inclusion of WHT and OAT insoluble fibres in the relevant quantities to obtain the associated EFSA health claims), fermentability was optimised by taking a weighted average of the fermentable fibres in question (weighted proportionally to their average SCFA production rate).

### Example 4: Validation of an In Vivo Method for Assessing SCFA Production

The plasma SCFA pharmacokinetics were assessed using the following protocol, to assess whether the compositions of the invention were actually fermented into SCFA in a subject.

Since one cannot measure SCFA production directly in vivo with existing technology, many studies have used concentrations of SCFAs present in stool samples collected from fibre-intervention study participants as indications of quantities of SCFAs produced in these same participants' guts, but these data are inaccurate at best. In particular, stool SCFA concentrations depend on the balance between the quantities of SCFAs produced and the quantities of SCFAs absorbed by the host.

In order rigorously study the underlying fermentation process in in vivo cohorts and measure the impact of our mixes on SCFA production in clinical trials, plasma SCFAs were measured here. Since SCFAs are detectable in the plasma but at far smaller concentrations, the inventors developed a highly sensitive LC-MS quantification method for SCFAs (cf. Methods).

This method was applied to blood samples collected every 20 minutes (for the first 8 hours) and then every hour (for the following 16 hours) following the ingestion of specific meals. N=3 trial participants were cycled through the following meals:
a. High-fibre, Mediterranean meal
b. High fat and high sugar junk food meal
c. Meal B+20 g of a fibre supplement (chosen between inulin, resistant maltodextrin and resistant dextrin, as a function of the patient's fermentation capability, determined using the ex vivo method described in Example 1)

Each meal was preceded by a 12 h fasting period and followed by a 24 h period of blood sampling without further consumption of food. d

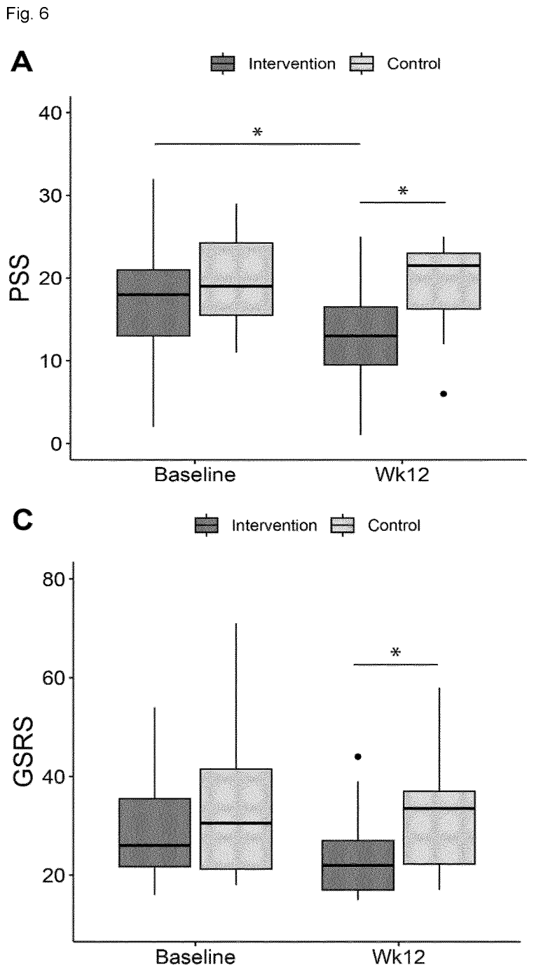

The results are presented in [FIG. 6] and [FIG. 7]. Accumulation of SCFAs in the blood is clearly seen. Spikes in butyrate can be observed post-meal A (high fibre Mediterranean diet meal) for participants 1 and 3, and after meal C (junk with fibre supplement) for participant 2 (FIG. 7). These data indicate that plasma SCFAs are valuable clinical endpoints and a good proxy for measuring SCFA production in real-time to test the clinical response to our fibre mixes.

### Example 5: Clinical Trial

### Methods: Study Design

This study was a two-arm, individually randomised, single-blinded, placebo-controlled clinical trial using an equal allocation ratio. The study was approved by Berkshire B NHS Human Research Ethics Committee (reference: 22/SC/0363). The study was registered on ClinicalTrials.gov (registration number: NCT05593926).

### Methods: Study Population

Written informed consent was obtained from all participants in accordance with the Declaration of Helsinki. Individuals with a primary diagnosis of pre-diabetes, but not receiving treatment for type 2 diabetes, were recruited across two London clinics. Inclusion criteria were: men and postmenopausal women aged 18-70 years of age, capacity to give informed consent, body mass index (BMI) of at least 25 kg/m2, baseline HbA1c (glycated haemoglobin) result within the range 5.8% (40 mmol/mol) to 6.5% (48 mmol/mol) within the past 12 months, willing to complete study requirements, and have access to a smartphone or computer. Exclusion criteria were: receiving medication to treat type 1 or type 2 diabetes in the previous 6 months, a BMI>45 kg/m2, loss of more than 5% body weight in the last 3 months, current participation in weight loss program or planned in the next 16 weeks, steroid use (except for over the counter NSAIDs, topical steroids, and inhalers), severe hepatic diseases, continuous antibiotic use for >3 days within 4 weeks prior to enrolment, continuous use of weight-loss drug within 3 months of study enrolment, gastrointestinal surgery (except for appendicitis or hernia surgery or co-existing pathology (Crohn's disease, coeliac disease, endometriosis, prostate cancer)), severe mental illness within 6 months prior to enrolment, receiving drug therapy to treat cholecystitis, peptic ulcers, urinary tract infection, acute pyelonephritis, urocystitis or hyperthyreosis, pituitary dysfunction, severe organic diseases including cancer, coronary heart disease, myocardial infarction or cerebral apoplexy, infectious diseases, including pulmonary tuberculosis and AIDS, history of alcoholism or substance misuse, significant dyslipidemia, severe hypertension (>160/100 mmhg), use of any food supplements to control blood glucose (e.g., chromium picolinate) within two months of study enrolment. Eligible participants were identified through GP practices, and via social media campaigns. Participants were withdrawn from the trial if they started medications used to control their pre-diabetes.

### Methods: Intervention

Consenting participants who met the eligibility criteria and completed the baseline assessment were individually randomised into one of the two groups, using a web-based system (Sealed Envelope) at a ratio of 1:1, stratified by sex and BMI. For 24 weeks, the intervention group consumed 20 g per day of

Composition 2, as described in [Table 1] and the placebo group received 2 g per day of cellulose. Both the intervention and placebo supplements were powdered and unflavoured, and were given to the participants in single serve sachets. The supplements were taken at any time of the day, consumed with water or mixed into any food or drink (e.g., water, breakfast cereal, tea). To enhance compliance, participants received a daily text message, asking them to confirm that they had taken the supplement. If they failed to consume the supplement on any given day, they were prompted to provide a reason.

### Methods: Study Measurements

Participants attended a central clinic at baseline, week 16 and week 24, where blood pressure, blood samples, and the Oral Glucose Tolerance Test for Insulin Sensitivity (ISI-OGTT) was performed. Fasting blood samples were performed after participants completed a 10-12 hour overnight fast. For the ISI-OGTT, blood samples were taken at −15, 0, 30, 60, 90, and 120 min for the measurement of plasma glucose and insulin concentrations. The insulin sensitivity index from the ISI-OGTT was calculated according to the equation derived by Matsuda & De Fronzo (1999) in which insulin sensitivity is estimated by dividing a constant (10,000) by the square root of the product of fasting glucose (FBG) times, fasting insulin (FBI) times, mean glucose (MG) times and mean insulin (MI). ISI-OGTT=10.000/(FPG*FPI)(MG*MI). The index is strongly correlated with insulin sensitivity derived using the euglycaemic clamp technique (Matsuda & De Fronzo, 1999).

### Methods: Primary and Secondary Endpoints

The primary endpoint was the change in HbA1c from baseline to week 16. Key secondary endpoints included changes in insulin sensitivity (measured using ISI-OGTT), FBI, blood lipids (cholesterol [total, LDL, HDL], triglycerides), inflammatory biomarkers (IL-6, IL-8, IL-10, CRP, TNF-α), and diastolic and systolic blood pressure from baseline to 16 and 24 weeks.

### Methods: Tolerability and Safety

Participants were asked to record whether they experienced any side-effects or medical events since starting the Intervention or Placebo, recorded via online surveys at the end of weeks 1-4, and on a monthly basis for the remainder of the trial. If they answered yes, adverse events were documented, reported, and reviewed for relatedness and expectedness within 24 hours.

### Methods: Statistical Analysis

Data were analysed using Python software. Statistical analyses were performed based on an intention-to-treat protocol. For the primary endpoint, an unpaired t-test was used to compare the percent change in HbA1c levels from baseline to 16 weeks between intervention and placebo. Two-way repeated measures ANCOVA with one within-subject factor (time) and one between-subject factor (randomisation) were used to compare secondary endpoints at baseline, 16, and 24 weeks, including HbA1c, insulin sensitivity, fasting blood insulin, lipid profiles (total cholesterol, LDL, HDL, triglyceride levels), inflammatory biomarkers (IL-6, IL-8, IL-10, CRP and TNF-α), and diastolic and systolic blood pressure. Post-hoc one-tailed paired t-tests were performed on significant main or interaction effects to compare differences between groups over time. For non-normally distributed outcome variables, robust ANCOVA and post-hoc comparisons with trimmed means were used. Subgroup analyses included the effects of baseline HbA1c levels on the primary and secondary endpoints. Effects with p-values<0.05 were considered statistically significant.

### Results

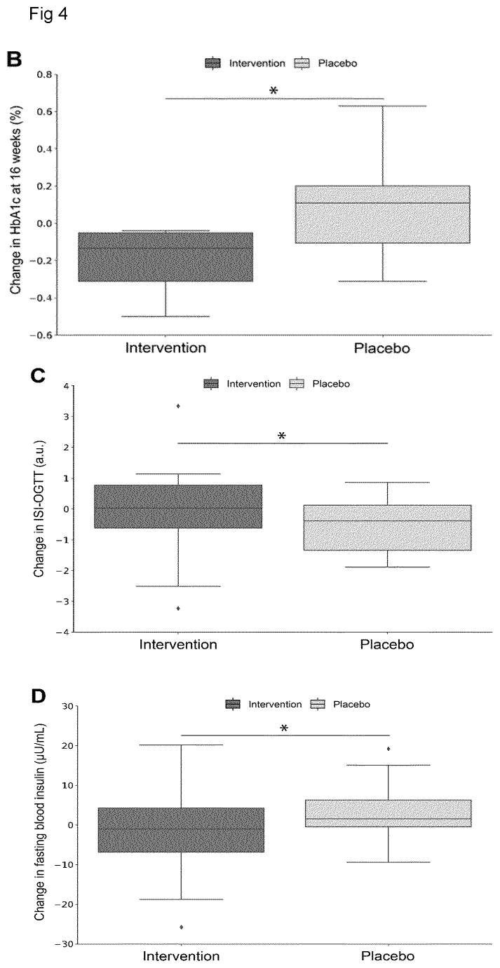

The results are provided in [FIG. 4]. Parametric t-tests identified a significant reduction in fasting blood insulin levels (p=0.04), and significant improvements in insulin sensitivity (p=0.03) in participants who consumed the fibre composition of the invention. HbA1c (%) in participants with lower baseline HbA1c levels (<6.0%) was significantly lower in participants who consumed the fibre composition of the invention (p=0.009).

### Example 6: Real World Microbiome Kit Data from Customers

### Methods: Stool Sample Collection and Processing

Volunteer customers who purchased a microbiome testing kit in accordance with Applicant's terms and conditions were included in this analysis. Participants were provided with a stool sampling kit, including a sample collection tube containing nucleic acid stabilisation buffer, a sample swab and instruction card, to use at home. Samples were then processed as follows. Stool DNA extraction was performed by homogenisation of sample (3000 rpm, 2 minutes) prior to removal of 200 μL of sample to be subjected to NA extraction on the kingfisher flex 96 system (origin: Thermo Fisher Scientific). Primers targeting the V4 region of the 16S gene were used. 16S rRNA sequencing was performed on an Illumina MiSeq platform.

### Methods: 16S Data Processing and Analysis

Demultiplexed fastq files were processed using default settings within QIIME2 2020.2 (https://qiime2.org) (37). Amplicon Sequence Variants (ASVs) were generated by denoising with DADA2. For taxonomic structure analysis, taxonomy was assigned to ASVs using a pre-trained Naïve Bayes classifier and the q2-feature-classifier plugin against the Silva 16S rRNA gene sequencing database. Samples were rarefied to a read depth of 10000 for diversity analyses. ANCOVA (analysis of covariance) was used to test for group differences in Shannon diversity index and Chao1 index accounting for the effects of body mass index (BMI). Beta-diversity, assessed using unweighted UniFrac distance (Pac Symp Biocomput. 2012:213-224), was used to compare groups using qiime2 plugins PERMANOVA (Permutational multivariate analysis of variance) and adonis. Significant features were corrected for multiple comparisons using the Benjamini-Hochberg FDR procedure, with corrected values of p<0.05 and q<0.25 considered statistically significant.

### Methods: Statistical Tests

Data were analysed using R software. Statistical analyses were performed based on an intention-to-treat analysis. However, as a sensitivity analysis, primary and secondary endpoints were analysed using the per-protocol population to investigate whether conclusions are sensitive to assumptions regarding the pattern of missing data. Group differences in baseline characteristics were assessed using χ2-tests for categorical variables and parametric t-tests or Wilcoxon signed-rank test for quantitative variables. For all comparisons, we tested the normality and homoscedasticity. Between group differences were analysed by two-way repeated measures ANOVA assessing the main effects of time and treatment. Within group analyses were performed using

Tukey post-hoc test when a main effect or interaction (time x treatment) were significant (p<0.05).

### Results

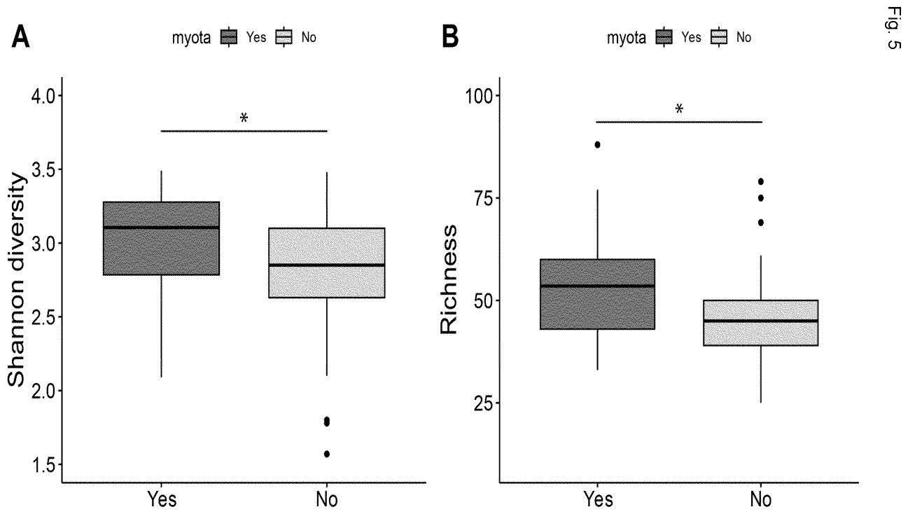

The Results are provided in [FIG. 5].

Wilcoxon signed-rank test identified a significant difference in Shannon diversity (p=0.007) and richness (p=0.03) in participants who consumed 300 g (or part of such dose) of Composition 2 as described in [Table 1] within a three-months period prior to submitting a stool sample (dark grey), compared to those who did not (light grey).

The multivariate statistical framework, MaAsLin2, implemented in R, was used to assess the relationship between intake of the fibre mix of the invention with microbial abundance (collapsed at genus level). Features were included if they had at least 10% non-zero values (across samples) and a minimum relative abundance threshold of 0.0001, both validated parameter settings in MaAsLin2. Significant features were corrected for multiple comparisons using the Benjamini-Hochberg FDR procedure, with corrected coefficient values of p<0.05 and q<0.25 considered statistically significant. [FIG. 5] shows that a significant difference in microbiota diversity is observed between the participants who consumed the fibre mix of the invention compared to participants who did not.

### Example 7: Prebiotic Intervention for Metabolic and Mental Health

### Methods: Study Design and Participants

This study was part of a larger 12-week open-label parallel RCT, comparing a control group who received healthy eating advice only, to an intervention group who received healthy eating advice plus a diverse prebiotic supplement in free-living patients with MetS. The analysis performed in this study is based on secondary outcomes from the ethics protocol submitted. The study was approved by the NHS Human Research Ethics Committee of City and East (reference: 23/LO/0515). Participants provided written informed consent in accordance with the Declaration of Helsinki. Briefly, individuals recruited for the study had at least 3 symptoms consistent with MetS, yet not receiving medication for their symptoms. Participants were randomly allocated to either the prebiotic fibre intervention or control. The prebiotic supplement has not been studied formally in a cohort of participants with MetS. It was therefore felt that more participants in the prebiotic arm (2:1) would yield more information about potential tolerance and side effects associated while controlling cohort size for practical purposes.

### Methods: Prebiotic Intervention

The prebiotic fibre group consumed 10 g per day of Composition 1 as described in [Table 1]. The prebiotic supplements were powdered and unflavoured, and were given to the participants in 300 g packets lasting for 30 days. A 10 g scoop was included in each packet and participants were advised to consume one level scoop once a day, at any time of the day. Participants could mix the supplement into water or stir it into food or drinks (e.g., breakfast cereal, coffee, tea). To minimise participant withdrawal and ensure consistent use of supplements, they received a weekly survey, asking them to confirm that they had taken the supplement on each day of the week. If they failed to consume the supplement on any given day, they were prompted to provide a reason.

### Methods: Dietary Recommendations

Both intervention and control groups were given dietary advice prior to starting the 12-week intervention period. Dietary recommendations were provided in written format to the participants, and were consistent with the Heart UK “Healthy Eating Guide” (found here: https://www.heartuk.org.uk/downloads/health-professionals/publications/healthy-eating-guide.pdf). Briefly, these recommendations emphasised a Mediterranean diet, rich in fruit, vegetables, and healthy fats (omega-3 fatty acids), while reducing refined sugar, salt, processed foods, and alcohol intake.

### Methods: General Demographics, Dietary Fibre Intake, and Gastrointestinal Symptoms Surveys

At baseline and week 12, participants answered questions about general demographics and health, daily dietary fibre (16-item Fiberscreen) and gastrointestinal symptoms (Gastrointestinal Symptom Rating Scale, GSRS).

### Methods: Neurocognitive Assessments

Neurocognitive assessments included the Hamilton and Montgomery Anxiety (HAM-A), Generalised Anxiety Disorder (7-item) (GAD-7), Patient Health Questionnaire (PHQ), and the Depression, Anxiety, and Stress Scale (42-item) (DASS-42).

### Methods: Blood Sample Collection and Processing

At baseline and week 12, participants completed a finger prick blood test. Finger-prick blood sampling kits were shipped to the participant's chosen address, with written instructions on correct usage. Blood samples are taken after an 8-hour minimum fast. Participants returned their sample in the mail using a prepaid envelope. Serum Separator Tubes (SST) were centrifuged to separate the blood serum within the sample for testing. Hs-CRP profiles were tested via a Roche Cobas c503 platform.

### Methods: Statistical Tests

Data were analysed using R software. Statistical analyses were performed based on an intention-to-treat analysis. Group differences in baseline characteristics were assessed using χ2-tests for categorical variables and parametric t-tests or Wilcoxon signed-rank test for quantitative variables. For all comparisons, we tested the normality and homoscedasticity. Between group differences were analysed by two-way repeated measures ANOVA assessing the main effects of time and treatment. Within group analyses were performed using Tukey post-hoc test when a main effect or interaction (time x treatment) were significant (p<0.05).

### Results

The results are provided in [FIG. 6]. Two-way repeated measures ANOVA identified a significant main effect of treatment for Perceived Stress Score (PSS) (F_{(1,87)}=6.34, p_{corr}=0.01) and Gastrointestinal Symptom Rating Scale (GSRS) scores (F_{(1,87)}=9.81, p_{corr}=0.002). Tukey's HSD post-hoc tests identified significant differences between the Intervention and Control groups at week 12 for PSS (p_{corr}=0.04) and GSRS (p_{corr}=0.02), and between baseline and week 12 PSS scores for the Intervention (p_{corr}=0.01), with lower scores for the intervention group. These results demonstrate the efficiency of the fibre mix of the invention to reduce stress and gastrointestinal symptoms.

1. A dietary fibre composition comprising a mixture of the following dietary fibres:
a. isolated fructo-oligosaccharides (FOS);
b. isolated inulin;
c. isolated resistant maltodextrin;
d. isolated resistant dextrin;
e. isolated galactomannan polysaccharide; and
f. isolated partially hydrolysed galactomannan.

2. The dietary fibre composition according to 0, wherein:
a. fructo-oligosaccharides are present in an amount of 10 to 50 wt %, based on the total weight of the composition;
b. inulin is present in an amount of 5 to 40 wt %, based on the total weight of the composition;
c. resistant maltodextrin is present in an amount of 3 to 30 wt %, based on the total weight of the composition;
d. resistant dextrin is present in an amount of 2 to 20 wt %, based on the total weight of the composition;
e. the galactomannan polysaccharide is present in an amount of 0.5 to 10 wt %, based on the total weight of the composition; and
f. partially hydrolysed galactomannan is present in an amount of 1 to 20 wt %, based on the total weight of the composition.

3. The dietary fibre composition according to claim 1, further comprising isolated galacto-oligosaccharides.

4. A The dietary fibre composition according to claim 1, further comprising at least one source of arabinoxylan and/or at least one source of beta-glucan.

5. The dietary fibre composition according to claim 4, wherein the source of arabinoxylan and/or the source of beta-glucan comprises wheat and/or oat fibre.

6. The dietary fibre composition according to claim 1, wherein the galactomannan polysaccharides is guar gum and/or wherein the partially hydrolysed galactomannan is partially hydrolysed guar gum.

7. A nutritional composition comprising the dietary fibre composition according to claim 1.

8. The nutritional composition according to claim 7, in the form of a drink, a food product or a nutritional supplement.

9. The nutritional composition according to claim 7, wherein said nutritional composition does not comprise any probiotic microorganism.

10. A method for preparing a nutritional composition, the method comprising admixing a dietary fibre composition according to claim 1, with other ingredients of a nutritional composition.

11. (canceled)

12. A method for increasing the production rate of at least one short chain fatty acid (SCFA) by the gut microbiota of a subject, the method comprising administering the dietary fibre composition according to claim 1 or a nutritional composition comprising the dietary fibre composition according to claim 1 to the subject.

13. The method according to claim 12, wherein the at least one SCFA is selected from acetate, propionate, butyrate, and a mixture thereof.

14. A method for providing at least one of the following therapeutic effects:
a. improving bowel function;
b. reducing gastrointestinal symptoms;
c. increasing microbiota diversity;
d. improving post-prandial glycaemic responses;
e. lowering blood lipids;
f. preventing elevation of blood cholesterol levels;
g. lowering blood glucose levels in an individual;
h. preventing elevation of blood glucose levels;
i. reducing blood pressure;
j. preventing the elevation of blood pressure;
k. improving intestinal homeostasis;
l. improving energy metabolism;
m. preventing obesity and/or overweight;
n. improving insulin sensitivity;
o. preventing, treating or reducing the severity of type 2 diabetes;
p. preventing, treating or reducing inflammation;
q. enhancing intestinal barrier function;
r. enhancing mucosal immunity;
s. preventing, treating or reducing the severity of an infection;
t. enhancing the bidirectional communication between the central and the enteric nervous system (gut brain axis);
u. preventing or reducing the severity of autism-spectrum, anxiety and/or a depressive behaviour;
v. preventing, treating and/or reducing the severity of irritable bowel syndrome;
w. reducing stress; and/or
x. reducing fasting blood insulin levels;
in a subject,
the method comprising administering the dietary fibre composition according to claim 1 or a nutritional composition comprising the dietary fibre composition according to claim 1 to the subject.

15. The method according to claim 14, wherein the therapeutic effect is achieved by increasing the production rate of at least one short chain fatty acid (SCFA) by the gut microbiota of an individual consuming the dietary fibre composition or the nutritional composition.

16. The method according to claim 15, wherein the SCFA is selected from acetate, propionate, butyrate, and a mixture thereof.

17. The method according to claim 16, wherein the SCFA is butyrate.

18. The method according to claim 13, wherein the SCFA is butyrate

19. The dietary fibre composition according to claim 2, wherein:
a. fructo-oligosaccharides are present in an amount of 10 to 40 wt %, based on the total weight of the composition;
b. inulin is present in an amount of 8 to 30 wt %, based on the total weight of the composition;
c. resistant maltodextrin is present in an amount of 3 to 25 wt %, based on the total weight of the composition;
d. resistant dextrin is present in an amount of 3 to 15 wt %, based on the total weight of the composition;
e. the galactomannan polysaccharide is present in an amount of 1 to 7 wt %, based on the total weight of the composition; and
f. partially hydrolysed galactomannan is present in an amount of 2 to 15 wt %, based on the total weight of the composition.

20. The dietary fibre composition according to claim 19, wherein:
a. fructo-oligosaccharides are present in an amount of 14 to 34 wt %, based on the total weight of the composition;
b. inulin is present in an amount of 10 to 26 wt %, based on the total weight of the composition;
c. resistant maltodextrin is present in an amount of 5 to 15 wt %, based on the total weight of the composition;
d. resistant dextrin is present in an amount of 4 to 12 wt %, based on the total weight of the composition;
e. the galactomannan polysaccharide is present in an amount of 1 to 5 wt %, based on the total weight of the composition; and
f. partially hydrolysed galactomannan is present in an amount of 5 to 12 wt %, based on the total weight of the composition.
